(19) United States
(12) Patent Application Publication
(10) Pub. No.: US 2004/0064151 A1
(43) Pub. Date: Apr. 1, 2004

(54) Ultrasonic forceps

(75) Inventor: Kenneth H. Mollenauer, Saratoga, CA (US)

Correspondence Address: Crockett & Crockett, 24012 Calle De La Plata, Suite 400, Laguna Hills, CA, 92653, US

(73) Assignee: Starion Instruments Corporation

(21) Appl. No.: 10/256,950

(22) Filed: Sep. 27, 2002

(51) Int. Cl. A61B 17/28

(52) U.S. Cl. 606/205

(57) ABSTRACT

Ultrasonic forceps adapted for use in open surgical forceps. The device is provided in the form of traditional open surgery forceps or tweezers, and the transmitting rod which transmits ultrasonic vibration from a proximally located transducer to the distally mounted welding horn runs through a lumen in one of the arms of the forceps.

### FIELD OF THE INVENTIONS

The inventions described below relate the field of open surgery and devices for performing surgery.

### BACKGROUND OF THE INVENTIONS

Presently available ultrasonic tissue cutters are designed for endoscopic use, and are generally provided in a configuration comprising a 5 or 10 mm diameter shaft, about 35 cm long, with a proximal handle and grasping jaws mounted on the distal tip of the shaft. These devices are designed to be inserted into an endoscopic workspace through a cannula, and to be operated through trigger mechanisms mounted on the handle. These devices, while well suited for endoscopic surgery, are ill-suited for open surgery as they interfere with the surgeons natural mode of handling devices in the open surgical field. The benefits of the ultrasound forceps may be obtained in the open surgical procedures by adapting the devices so that they conform to the typical form of other devices used during open surgery.

The forceps currently in use incorporate two symmetrical and equally flexible grasping arms (also referred to as blades). Symmetric and equally flexible blades are generally desirable because it is easier and cheaper to build these devices. However, for ultrasonic forceps, flexibility in the blade may cause power transmission losses (during transmission from the forceps into the tissue) and heat loss through the handle and blade. The heat loss can cause injury to the patient, in anatomical structures near the desired point of application, and can cause discomfort or injury the surgeon.

### SUMMARY

The devices and methods described below provide for open-surgery ultrasonic forceps operable to divide and seal body tissue during surgical operations. The forceps include a rigid straight blade matched to an opposing flexible, compliant or pivoting blade. The first blade can be non-pivoting, constructed as an anvil upon which the second blade acts, or the both blades may pivot, while the first blade is rigid and the second blade flexes significantly more than the first blade when the forceps are closed upon body tissue, or both blades may be rigid while the spring force of closure is limited by the strength of the pivot joint upon which they rotate. The blades are sized and dimensioned so that they are operable by direct manipulation of the blades by the surgeon, in the manner typically used to operate open-style forceps.

### BRIEF DESCRIPTION OF THE DRAWINGS

FIG. 1 shows open-style forceps with an ultrasonically driven grasping tip.

FIG. 2 shows open-style forceps with an ultrasonically driven grasping tip.

### DETAILED DESCRIPTION OF THE INVENTIONS

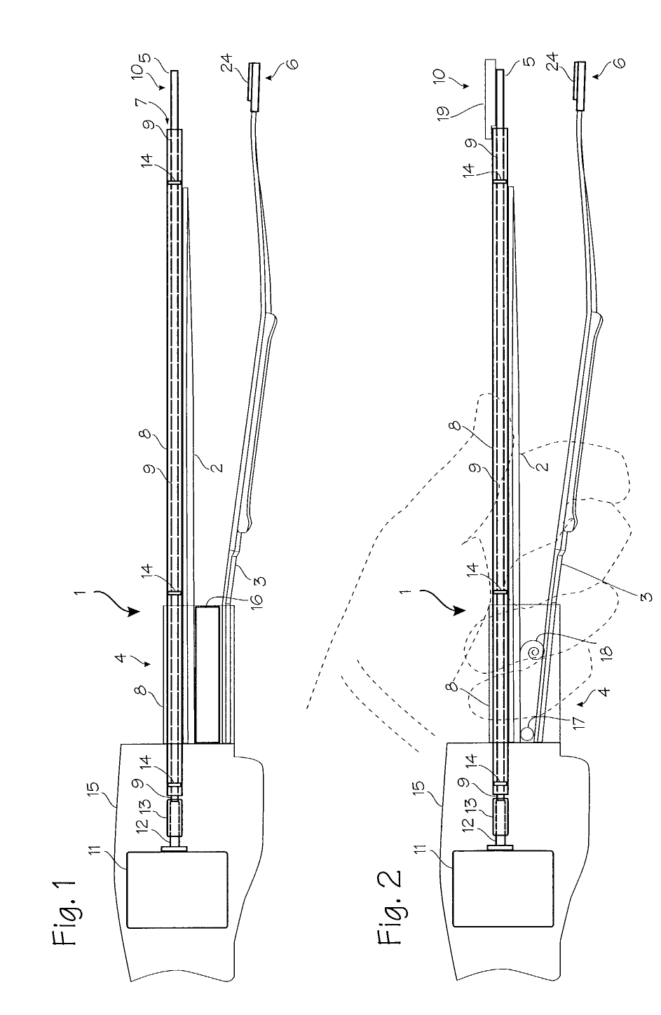

FIG. 1 shows open-style forceps with an ultrasonically driven grasping tip. The term “forceps” refers to a specialized surgical instrument resembling tongs or tweezers, used for grasping and moving body tissue during surgery. This pair of forceps has been modified by the addition of mechanisms within the handle and tips that are operable to heat tissue held between the tips. The degree of heating is controlled and limited to heat tissue to the degree necessary to seal and cauterize the tissue and permit dividing the tissue upon application of pressure, but it may also heated to higher temperatures needed to cut the tissue held between the grasping tips without application of significant pressure.

The forceps 1 include two grasping arms 2 and 3 fixed relative to each other at the proximal joint 4. The grasping arms may be fixed to each other or fixed to an intervening resilient block or spring, and may be referred to variously as blades, tongs, tines or arms. The forceps may also be referred to as tweezers or tongs, and are distinguishable from endoscopic graspers and the like by the absence of structure intermediate the surgeons hand and the forceps arm. The grasping arms are sized and dimensioned so that they are operable by direct manipulation of the blades by the surgeon, in the manner typically used to operate open-style forceps. To that end, the forceps arms are preferably about 8 inches (40 cm) long and are spaced from each other by about one inch (2.5 cm) at the tips. The forceps may be made in a broader range of sizes which are comfortably operated by hand, and which are suitable for various open surgical tasks. The grasping arms may be made in the broad range of about 4 to 12 inches long, with the tip gap in the rang of 0.5 to 2 inches.

The first grasping arm 2 is substantially straight and rigid, while the second grasping arm 3 may be flexible and curved as desired to permit closure of distal tips 5 and 6. A lumen 7 extends from the proximal end of the first tong to the distal end. The lumen may be formed, as shown, by the interior of the tube 8 which is fixed to grasping arm 2, or the grasping arm may be formed with a lumen running through the tong. A rod 9 is disposed within the lumen, and extends distally from the lumen so that the distal segment 10 is exposed and may serve as a grasping tip of the forceps. The distal segment serves as an ultrasonic welding horn. The rod and distal segment is made a hard, light, and thermally conductive materials such as metal (steel or titanium or the like), or carbon fiber or rigid plastics, and will become hot in use because it will be rapidly rubbed against body tissue. The rod may even be hollow, rather than solid. The distal segment has a cylindrical cross section, with a diameter of about 1 to five millimeters.

At its proximal end, the rod is mechanically fixed to the transducer 11 so that mechanical vibration of the transducer is translated into mechanical vibration of the distal segment 10. The transducer may be releasably attached to the rod with the pin 12 which is screwed or press-fit into the bore of a boss 13 fixed to the proximal end of the rod. The rod is held centrally within the lumen, to avoid contact with the inner wall of the lumen (so as to prevent heating of the tube), with several silicon rings 14. The transducer and the connections between the transducer and the rod (along with necessary electrical connections for powering the transducer and sensing temperature at the tip of the device, and any other control features) are housed in the proximal housing 15. Disposed between the proximal sections of the tongs, a spacing block 16 holds the tongs apart so as to position the tips 5 and 6 a predetermined apart from each other (preferably about one inch).

FIG. 2 illustrates the device of FIG. 1 modified in several respects. In this embodiment, the second grasping arm 3 is substantially rigid, and does not flex when squeezed by the surgeon. Instead, the grasping arm rotates about the hinge 17 which pivotally connects the first and second tongs at the proximal ends. A spring 18 is preferably included to bias the forceps in the open position. The spring is disposed between the grasping arms, near the proximal end of the grasping arms, and any suitable spring may be used. Also in FIG. 2, the distal segment of the rod which extends distally from the tube is covered on its outer side by the heat insulator in the form of the pad 19 (which may be made of PTFE or silicone or other suitable material). The pad serves to protect body tissue outside the grasping tips from heat generated by the forceps tip during operation.

The second grasping arm 3 is preferably a flexible, flat forceps blade which is curved to bias the tips 5 and 6 away from each other while permitting the second grasping arm to be deformed and flexed easily by the surgeon to bring the tips together. On the distal tip 6 of the second grasping arm, a resilient pad 24 is disposed on the grasping face opposing the grasping face of the distal segment of the rod. The pad may be grooved, toothed, or ridged to assist in holding tissue.

A contact relay or other switch or sensor may be disposed between the grasping arms, spaced relative to the grasping arms such that closure of the grasping arms to bring the grasping tips together also brings the grasping arms into contact or interaction with the switch or sensor, so that the transducer is operated in response to closure of the forceps.

In general, the device may be described as a pair of forceps or tweezers characterized by a first arm and a second arm, where each of arm has a proximal end and distal end, each arm has a gripping face disposed on the distal end that define surfaces generally perpendicular to the plane defined by the grasping arms. These gripping surfaces are movable into apposition with each other upon closing of the tweezers. The tweezers are closed while the tweezer arms are held in the surgeons hand. As shown in FIG. 2, the surgeon squeezes directly on the tweezer arms with his fingers and thumb, without need for any intervening mechanism. The pivot point for the tweezers lies in the housing 15, which is located proximally of the surgeons hand and the point of application of operating force. Thus, the forceps are sized and dimensioned such that, in use, the point of closure at the grasping tips is distal of the surgeons hand and the force receiving members (the grasping arms), while the pivot point of the grasping arms is proximal to the hand, opposite the grasping tips.

The transducer is selected to provide significant power to cause vibration or small displacement reciprocation of the rod. Transducer sufficient to provide power output at the tip of 1 to 30 watts (as measured by the amount of energy transferred to a small volume (5 cc) of water) may be used to drive the rod in the device illustrated in Figures, but the transducer power required will vary with variations in device size, rod weight, and other design parameters. The transducer may be driven by various control circuits and power supplies, such as the Ultracision™, Autosonic™, or Axyaweld™ ultrasound generators which are commercially available. Any such means for driving the transducer to cause vibration of the distal segment of the rod may be used.

The ultrasonic forceps can be used in most any open surgical procedure in which a surgeon would want to divide and/or seal tissue. In use, the forceps are plugged into a control box that provides suitable electrical stimulation to the transducer. The forceps are held in the hand of the surgeon, and the surgeon manipulates the blades and body tissue within an open surgical field to bring the body tissue between the blades. When a mass of body tissue which is to be sealed is located between the blades and the grasping faces, the surgeon then squeezes the forceps arms together by hand to press the tips toward each other and trap the body tissue between the grasping faces. Either through action of the contact relay or through a separate switch, the surgeon activates the transducer to cause rapid vibration of the rod 9, so that the distal segment vibrates rapidly against the body tissue between the grasping faces. Vibration of the distal segment against body tissue heats the body tissue to the degree necessary to heat and seal the body tissue.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

I claim:

1. A surgical device comprising:
tweezer-style forceps comprising a handle section, a first grasping arm and a second grasping arms extending from the handle section;
said first grasping arm being substantially rigid, said first grasping arm having a lumen extending from the distal end to the proximal end thereof;
a rod extending through the lumen of the first grasping arm, said rod having a distal segment extending distally from the lumen to form a first grasping tip for the first grasping arm;
a transducer operably connected to the rod such that operation of the transducer causes vibration in the rod;
said second grasping arm being movably mounted to the handle, said second grasping arm having a second grasping tip disposed on the distal end;
said first and second grasping arms being operable by hand to bring the first and second grasping tips into opposition;
means for driving the transducer to cause vibration of the distal segment of the rod.

2. The device of claim 1, wherein the first grasping tip is characterized by a grasping face and an outer surface, and the second grasping tip is characterized by a grasping face and an outer surface, and wherein the device further comprises:
a resilient surface on the grasping face of the second grasping arm.

3. The device of claim 1, wherein the first grasping tip is characterized by a grasping face and a outer surface, and the second grasping tip is characterized by a grasping face and an outer surface, and wherein the device further comprises:
a insulating cover disposed over the outer surface of the first grasping tip.

4. The device of claim 1 wherein the distal segment of the rod is cylindrical and has a diameter of 1 to 5 mm.

5. The device of claim 2 wherein the distal segment of the rod is cylindrical and has a diameter of 1 to 5 mm.

6. The device of claim 3 wherein the distal segment of the rod is cylindrical and has a diameter of 1 to 5 mm.

7. An ultrasonic surgical apparatus comprising:
a housing;
a transducer carried by the housing for generating ultrasonic vibration;
tweezers comprising a first blade and a second blade, wherein the first blade is substantially rigid, and the second blade is flexible and operable to be forced by hand toward the first blade;
a first grasping tip extending from the first blade and a second grasping tip extending from the second blade;
a rod extending through the first blade, said road coupled to the ultrasonic element for receiving ultrasonic vibration therefrom and transmitting the vibration to the first grasping tip.

8. The device of claim 7, wherein the first grasping tip is characterized by a grasping face and an outer surface, and the second grasping tip is characterized by a grasping face and an outer surface, and wherein the device further comprises:
a resilient surface on the grasping face of the second grasping arm.

9. The device of claim 7, wherein the first grasping tip is characterized by a grasping face and a outer surface, and the second grasping tip is characterized by a grasping face and an outer surface, and wherein the device further comprises:
a insulating cover disposed over the outer surface of the first grasping tip.

10. The device of claim 7 wherein the distal segment of the rod is cylindrical and has a diameter of 1 to 5 mm.

11. The device of claim 8 wherein the distal segment of the rod is cylindrical and has a diameter of 1 to 5 mm.

12. The device of claim 9 wherein the distal segment of the rod is cylindrical and has a diameter of 1 to 5 mm.

13. A medical device comprising:
a pair of tweezers characterized by a first arm and a second arm, each of said arm having a proximal end and distal end, said first arm having a first gripping face disposed on the distal end thereof, said second arm having second gripping face disposed on the distal end thereof, said gripping faces defining surfaces generally perpendicular to a plane defined by the grasping arms, said surfaces being movable into apposition with each other upon closing of the tweezers;
a vibrating element disposed on the distal end of the gripping face of the first arm;
means for vibrating the vibrating element.

14. The device of claim 13, wherein the device further comprises a resilient surface on the second gripping face of the second grasping arm.

15. The device of claim 13 wherein the device further comprises a insulating cover disposed over the outer surface of the distal end of the second arm.

16. The device of claim 13 wherein the distal segment of the rod is cylindrical and has a diameter of 1 to 5 mm.

17. The device of claim 14 wherein the distal segment of the rod is cylindrical and has a diameter of 1 to 5 mm.

18. The device of claim 15 wherein the distal segment of the rod is cylindrical and has a diameter of 1 to 5 mm.

19. A medical device comprising:
a pair of forceps characterized by a first arm and a second arm, each of said arm having a proximal end and distal end, each of said arm being rotatably fixed to the other at a midpoint thereof, said first arm having a first gripping face disposes on the distal end thereof, said second arm having second gripping face disposed on the distal end thereof, said gripping faces defining surfaces generally perpendicular to a plane defined by the grasping arms, said surfaces being movable into apposition with each other upon closing of the forceps;
a vibrating element disposed on the distal end of the gripping face of the first arm;
means for vibrating the vibrating element.

20. The device of claim 19, wherein the device further comprises a resilient surface on the second gripping face of the second grasping arm.

21. The device of claim 19 wherein the device further comprises a insulating cover disposed over the outer surface of the distal end of the second arm.

22. The device of claim 19 wherein the distal segment of the rod is cylindrical and has a diameter of 1 to 5 mm.

23. The device of claim 20 wherein the distal segment of the rod is cylindrical and has a diameter of 1 to 5 mm.

24. The device of claim 20 wherein the distal segment of the rod is cylindrical and has a diameter of 1 to 5 mm.
